(19) United States
(12) Patent Application Publication
(10) Pub. No.: US 2011/0104279 A1
(43) Pub. Date: May 5, 2011

(54) HEALING POWDER AND METHOD OF USE THEREOF

(76) Inventors: Philip A. Marraccini, Harrison, NY (US); Maria Dioguardi, Harrison, NY (US)

(21) Appl. No.: 12/612,084

(22) Filed: Nov. 4, 2009

(51) Int. Cl. A61K 9/00; A61K 31/7016; A61K 35/24; A61K 31/715; A61K 38/16; A61K 31/722; A61K 31/718; A61K 31/685; A61K 31/70; A61P 17/02

(52) U.S. Cl. 424/484; 514/53; 424/537; 514/54; 514/55; 514/60; 514/78; 514/23; 514/17.2

(57) ABSTRACT

A method for treating wounds, comprising providing a dry powder mixture, which when partially hydrated, forms a gel having an osmotic pressure sufficient to achieve and maintain antiseptic conditions in hydrated portions of the gel, the gelled portion comprising a biocompatible polymer, the dry powder being readily wettable by wound secretions; and applying the dry powder to a wound having wound secretions in sufficient excess to produce a self-adherent gel cake having a dry powder surface. The administration may be repeated by administration of the dry powder while wound healing progresses.

### BACKGROUND OF THE INVENTION

1. Field of the Invention

The present invention relates to the field of wound dressings, and more particularly to powders adapted to be applied to a wound, which assist in wound healing.

2. Prior Art

U.S. Pat. No. 5,270,042, expressly incorporated herein by reference discloses wound healing compositions. See also, U.S. Pat. Nos. 3,943,248, 4,401,651, 4,504,470, 4,725,438, 4,883,664, 4,904,674, and 5,116,600, expressly incorporated herein by reference.

Cornstarch has been known as a soothing and drying agent for irritated skin, such as diaper rash. Typically, this is applied to intact skin. When cornstarch is used as an anti-sticking agent on surgical gloves, this can cause problems, both for patients, and for health care providers.

Normal cornstarch is a dextrin, and contains amylose and amylopectin. It is usually included as an anti-caking agent in powdered sugar (10× or confectioner's sugar). Cornstarch is used as a thickening agent in liquids. As the starch is heated, the molecular chains unravel, allowing them to collide with other starch chains to form a mesh, thickening the liquid. [12]. So-called waxy maize starch lacks amylase, and contains exclusively amylopectin.

Sugar pastes are known wound treatments.

The Dressings Times, Vol. 3, No. 2, discusses wound dressings which employ sugar pastes. (www.smtl.co.uk/WMPRC/DressingsTimes/vol3.2.txt). It reports that, in 1976, Herszage and Montenegro of Argentina used ordinary sugar to treat the wounds of two patients with post-surgical necrotic cellulitis. Further successes followed and in 1980 they reported on the use of sugar paste in 120 infected wounds and recorded a cure rate of 99.2%.[1] The time taken for the wounds to heal varied between 9 days and 17 weeks, but it was observed that odor and secretion began to diminish within 24 hours and disappeared totally after 72 to 96 hours of treatment. In 1985, Trouillet et al[2] described the use of sugar in the treatment of 19 patients with acute mediastinitis following cardiac surgery. Wounds were packed every 3 to 4 hours with ordinary commercially available granular sugar (sucrose). The authors noted near complete debridement followed by the rapid formation of granulation tissue and eradication of bacterial infection after an average of 7.6 days of treatment.

Sugar was first used as a dressing in Northwick Park Hospital in 1982 when it was placed into infected radical vulvectomy wounds that had not responded to more conventional therapies. However, due to the nature of such wounds, packing with granular sugar was found to be impossible and therefore a thick paste was developed. Other early patients to be treated with sugar at Northwick Park were two hypo-gammaglobulinaemic individuals who had developed extensive tracking sinuses. For these, a thin paste was formulated that could be injected into the narrow wounds.

Thick sugar paste has a consistency similar to that of modeling clay and can be molded in the gloved hand immediately prior to packing into cavities with large openings such as pressure sores. Thin sugar paste resembles thin honey; it is suitable for instillation into cavities with small openings with a syringe and fine plastic tube or catheter.

Formulae for sugar pastes (Prior Art)

| | |
|---|---|
| Thin | Thick |

| | | | | |
|---|---|---|---|---|
| Caster sugar | 1200 | g | 1200 | g |
| (fine granular sucrose) | | | | |
| Icing sugar - additive free | 1800 | g | 1800 | g |
| (powdered sucrose) | | | | |
| Polyethylene glycol 400 | 1416 | ml | 686 | ml |
| Hydrogen Peroxide 30% | 23.1 | ml | 19 | ml |

(Final concentration of hydrogen peroxide is 0.15% v/w.)

The pastes are prepared in the hospital pharmacy by combining the H₂O₂ with the PEG 400 and then incorporating this solution into the sugars with the aid of a mechanical mixer.

When homogenous the paste is packed into screw capped plastic containers and stored at 4 C. The pastes are chemically stable for at least 6 months from preparation.

Polyethylene glycol (PEG) 400 was chosen as the lubricant because it does not interact with other components of the paste and is used in a variety of pharmaceutical preparations. It is a synthetic polymer that is also used in the cosmetic industry and has significant anti-bacterial properties. [3][4] Polyethylene glycol 400 can be absorbed from mucous membranes and high blood levels may be nephrotoxic.[5] Although no toxic effects have been noted in our patients, many of whom are elderly and frail, sugar paste should be used with care in patients with impaired renal function as any absorbed polyethylene glycol is excreted renally.

Sugar paste has been used on most wound types but it has been found to be particularly effective for treating infected and malodorous wounds. Twice daily application are advised to provide the optimum antibacterial effect. This has been demonstrated both in patients with malodorous wounds (when the smell of infected necrotic tissue is removed after 2-3 days), and in patients with infected abscesses. Irrigation with thin sugar paste has achieved successful results in patients with chronic discharging sinuses who had previously failed to respond to other therapies. Repeated application over 3 to 6 weeks is generally required to bring about complete healing. Sugar paste lowers the pH of wounds to approximately 5 which may be important in infected wound although the paste does not stimulate or retard granulation tissue formation in clean wounds in the pig model.[6]

Sugar paste (thick and thin) is rapidly bactericidal against all organisms so far tested when challenged according to a modified British Pharmacopoeia antimicrobial preservatives effectiveness test. When samples of the paste were inoculated with Staphylococcus aureus, Streptococcus faecalis, Escherichia coli or Candida albicans, to give 10⁵ cfu/gram, less than 10 cfu/gram were detectable after 1 hour at 25 C.[7] Pastes diluted with serum have a reduced bactericidal effect −75% paste in serum gave an 80% reduction in viable numbers of S. aureus within 2 hours and a 99% reduction in viable numbers of Proteus mirabilis within 1 hour[3].

Honey is also known as a wound care agent. Studies suggest that the antibacterial and/or wound healing effects are not entirely due to the osmotic effects. Reports attribute at least aspects of any beneficial effect to bee enzymes and/or the botanical sources. [16]

See also, [12].

Although the application of sugar to a wound creates an environment with low water activity (aw) and high osmotic pressure, overall the wound remains moist. (The water activity of a solution is the ratio of its water vapor pressure to that of pure water at the same temperature so that aw=P/Po).

The effect of reducing water activity values on the growth of bacteria has been investigated by Chirife et al[8] who determined the limiting water activities at which different species of bacteria will grow. We have determined the water activity of our pastes, at different dilutions in serum, by measuring water vapor pressure at 25 C with an electronic hygrometer. Undiluted pastes have an almost zero availability of water because the sugar (sucrose) is dispersed in Polyethylene glycol 400 which does not contain water. Sugar has an osmotic action which can be thermodynamically related to water activity by the following equation: [8]

O=(RT/V)×log(1/aw),

where O is the osmotic pressure,

R is the gas constant,

T is the absolute temperature in degrees Kelvin,

V is the partial molal volume of water, and

aw is the water activity.

Thus, by determining water activity, the osmotic pressure can be calculated. From this equation it will be seen that a solution of low water activity has high osmotic pressure.

Because of the difficulty of conducting a controlled trial of sugar paste in human wounds, an animal study has been conducted[6] using a method similar to that reported by Winter and Scales.[9] Full thickness wounds 25 mm square, and 9 mm deep were made in the backs of pigs and around each was placed a colostomy stoma ring. This in turn was covered with a semipermeable plastic film dressing (Opsite) so as to form a moist chamber. Wounds were either covered with Opsite alone, or packed with thick sugar paste or cotton gauze soaked in various antiseptic solutions and then covered in Opsite. The results showed that there was no significant difference between wounds left unpacked, but covered with Opsite, and those Opsite covered wounds packed with sugar paste, indicating that although sugar paste did not stimulate the formation of granulation tissue, neither did it cause inhibition or toxicity. However, all wounds packed with antiseptics showed evidence of delayed healing, especially those containing chlorhexidine gluconate 0.2%. The pig model wounds were not infected so no conclusions can be drawn on the relative value of Opsite and sugar paste for healing infected wounds.

Dressing Times concluded that sugar paste should be considered for the management of all infected and malodorous wounds. It is a far less expensive alternative to Debrisan and similar products which are of dubious efficacy and are often difficult to remove from wounds. Sugar paste was also considered superior to charcoal dressings for treating malodorous wounds as it removes the cause of the smell and in this respect is similar to metronidazole gel. However sugar paste may be preferable to metronidazole gel for treating such wounds as the use of topical antibacterials and antibiotics should be avoided.[10]

Sugar paste lacks the toxicity of most antiseptics and it does not disrupt the architecture of the healing wounds, as does packing with gauze.

The paste is self-sterilizing and can be produced in different viscosities to suit all kinds of wound and it is not painful to apply. It may cause bleeding when granulation tissue is well formed, at which stage simple, non-impregnated dressings should be applied which will keep the wound moist and allow epithelialization to occur.

While there appears sufficient anecdotal and experimental basis to support the use of sugar pastes as a therapy for wound healing, the syrup dosage form poses its own problems, especially for mobile patients, but also for those who are sedentary or incapacitated. It is therefore desired to provide a formulation which achieves similar (or superior) osmotic antibacterial effects to those of a sugar syrup, while being administrable in a dry powder form. Likewise, a sugar syrup compels a dressing over the wound which retains the syrup in place, and thus does not permit “breathing” or drying of the wound. Aspects of the present invention therefore involve improvements in a formulation administrable as a dry antibacterial powder to a wound.

Collagen is the main protein of connective tissue in animals and the most abundant protein in mammals, making up about 25% to 35% of the whole-body protein content. It is naturally found exclusively in metazoa, including sponges. The gelatin used in food and industry is derived from the partial hydrolysis of collagen. Collagen has been widely used in cosmetic surgery, as a healing aid for burn patients, for reconstruction of bone and a wide variety of dental, orthopedic and surgical purposes. When used cosmetically, there is a chance of allergic reactions causing prolonged redness; however, this can be virtually eliminated by simple and inconspicuous patch testing prior to cosmetic use. This is less of an issue with hydrolyzed collagen, which is denatured and tends to be less antigenic, and is soluble. Most medical collagen is derived from young beef cattle (bovine) from certified BSE (Bovine spongiform encephalopathy) free animals. Porcine (pig) tissue is also widely used for producing collagen sheet for a variety of surgical purposes. Collagens are widely employed in the construction of artificial skin substitutes used in the management of severe burns. Although it cannot be absorbed through the skin, collagen is now being used as a main ingredient for some cosmetic makeup. [14]

Hydrolyzed collagen is usually made from type I or type III collagen by an enzymatic hydrolysis process. It is also called collagen hydrolysate, collagen peptide, gelatin, gelatin hydrolysate and hydrolyzed gelatin. Hydrolyzed collagen is mainly extracted from the bones and the skin from porcine, bovine and fish origin. The hydrolysis process allows to cut the collagen protein of about 300 000 Da, in small peptides having an average molecular weight comprise between 2000 and 5000 Da. The amino-acid content of hydrolyzed collagen is the same as collagen. Hydrolyzed Collagen contains 20 amino acids, including 8 out of 9 essential amino-acids (there is no tryptophan). It is characterized by the predominance of glycine, proline and hydroxyproline, which represents around 50% of the total amino-acid content. Glycine and proline concentration is 10 to 20 times higher than in other proteins. Hydrolyzed collagen is used in cosmetics since a very long time for its moisturizing properties. [15]. Orally administered hydrolyzed collagen apparently has beneficial effects on skin, and it is possible that during wound healing, external hydrolyzed collagen is catabolized and facilitates healing. A US FDA approved hydrolyzed collagen gel is available. [16]

### REFERENCES

1. Herszage L. et al., Tratamiento de las heridas supuradas con azucar granulado comercial, Biol Trab Soc Argent., 1980, 41, 315-330.
2. Trouillet J. L., et al., Use of granulated sugar in treatment of open mediastinitis after cardiac surgery, Lancet, 1985, 2, 180-183.
3. Ambrose U. An investigation into the mode of action of Northwick Park Hospital sugar pastes. Hatfield Polytechnic, 1986, B. Sc. Applied Biology Thesis.
4. Chirife J., et al., In-vitro antibacterial activity of concentrated polyethylene glycol 400 solutions, Antimicrob. Ag. Chemother., 1983, 24, 409-412.
5. Wilson C. G. and Thomas N. W. Interaction of tissues with polyethylene glycol vehicles Pharm. Int., 1984, 5 94-97.
6. Archer H. G. et al., A controlled model of moist wound healing: comparison between semi-permeable film, antiseptics and sugar paste. J. exp. Path., 1990, 75, 155-170.
7. Gordon H., et al., Sugar and wound healing Lancet, 1985, 2, 663-664.
8. Chirife J., et al., In-vitro study of bacterial growth inhibition in concentrated sugar solutions: microbiological basis for the use of sugar in treating infected wounds, Antimicrob. Ag. Chemother. 1983, 23, 766-773.
9. Winter G. D. and Scales J. T. Effect of air drying and dressings on the surface of a wound Nature, 1963, 197, 91-92.
10. Morgan D. Formulary of Wound Management Products (3^{rd} edition), 1989, Clwyd Health Authority, Preswylfa, Hendy Road, Mold, Clwyd CH7 1PZ.
11. www.peoplespharmacy.com/2007/09/17/sugar-speeds-wo/
12. en.wikipedia.org/wiki/Cornstarch
13. www.smtl.co.uk/WMPRC/DressingsTimes/vol3.2.txt
14. en.wikipedia.org/wiki/Collagen
15. en.wikipedia.org/wiki/Hydrolyzed_collagen
16. fanaticcook.blogspot.com/2007/06/honey-for-wound-healing.html

### SUMMARY AND OBJECTS OF THE INVENTION

The present invention comprises a dry powder wound dressing for application to broken or closed skin and mucous membranes, comprising a sufficient amount of a sugar, e.g., a monosaccharide or disaccharide (or in some cases, trisaccharides) to produce, when in contact with an open wound, a bacteriostatic or bacteriocidal environment, based on at least a high osmotic tension. The dry powder wound dressing also includes a gel matrix-forming component which is, in a dry state, compatible with the sugar such that normal levels of vibration or triboelectric forces do not tend to separate the components. The gel matrix-forming component may comprise, for example, one or more compositions selected from the group consisting of cornstarch, hydrolyzed collagen, natural collagen powder, synthetic collagen powder, mucopolysaccharides, pectin, or the like. The amount of the gel matrix-forming component is sufficient, when the formulation is wet with wound secretions over a range of clinical rates, to form a bacteriostatic or bacteriocidal gel which is self-adherent to the wound, and which is non-irritating and biologically compatible with normal healing.

It is generally preferable that the gel or cake be oxygen permeable, and not form a dense shell or barrier which results in anoxic conditions at the wound, or blocks wound secretions from release, resulting in a trapped liquid which can fester. Thus, would secretions are preferably wicked into the powder, and allowed to gel with dry or relatively dry portions of the cake. Thus, a free gel surface subject to air drying is to be generally avoided, since this could form a barrier which blocks oxygen permeation and leads to fluid accumulation.

The dry powder wound dressing may also contain polylactic acid, which is broken down or hydrolyzed into lactic acid.

The remainder of the dry powder wound dressing may be inert or otherwise compatible to form a pharmaceutically acceptable formulation

The dry powder may be provided in a resealable multi-dose shake container, gelatin capsule, single dose, single use pouch or bag, as part of a sterile dressing, and/or in a spray form (self-propelled or pump) of a powder suspended in a volatile liquid.

Preferably, the powder is sterile, and is for example treated with heat, ethylene oxide, radiation, ultraviolet light, or subjected to another suitable sterilization procedure. In some cases, sterility per se is not required, and commercial forms of the powder components may be employed, especially in view of the antiseptic action of the powder itself when moistened.

It is therefore an object of the invention to provide a wound healing powder, comprising a sugar selected from the group consisting of one or more pharmaceutically acceptable monosaccharides and disaccharides, in an amount sufficient, when wet with wound secretions, to ensure antiseptic properties of the powder. For example, the sugar may comprise at least 25% by weight of the powder mixture. The mixture also comprises a gel forming agent which forms a bioabsorbable biocompatible matrix with wound secretions. The gel-forming agent may, for example, be a polysaccharide and/or carbohydrate, a peptide and/or protein. The gel-forming agent may also be a copolymer, such as a copolymer formed of one or more of saccharide or saccharide derivative monomers and/or a glycosylated (or other saccharide) protein. The gel-forming agent(s) are biocompatible, and generally are bulk components of the powder, and for example is present in an amount of at least 25% by weight of the powder mixture. A further optional component is a surfactant, such as lecithin and/or chitosan-derivative, present in an amount sufficient to increase wettability of the powder, for example lecithin in a quantity of less than about 1% by weight.

It is another object of the invention to provide a method for healing a wound on skin or mucous membranes, comprising administering a pharmaceutically acceptable powder to the wound comprising a sugar selected from the group consisting of one or more pharmaceutically acceptable monosaccharides and disaccharides, in an antiseptic effective amount; and a gel-forming agent which forms a bioabsorbable biocompatible matrix with wound secretions, in an amount of, e.g., at least 25% by weight of the powder mixture, the powder being provided in sufficient quantity to form a cake on the wound from wound secretions and the powder. A non-toxic surfactant may be added to improve powder wettability.

The sugar may be present in an amount of at least 20% by weight and the gel-forming agent is present in an amount of at least 40% by weight. In a preferred formulation, the sugar is present in an amount of about 33% by weight and the gel-forming agent is present in an amount of about 50% by weight. The surfactant, if present, may be present in an amount of less than 1% by weight.

The sugar preferably comprises a finely powdered sucrose, e.g., 2×, 6×, 8×, 10×, 12×, for example, the sugar can be commercially available confectioner's sugar. However, as may be appropriate, any fineness and composition monosaccharide, disaccharide, sugar alcohol, or other osmotic agent may be employed. Thus, for example, 1× (normal granulated cane sugar) may be employed where appropriate, without departing from the spirit of the invention. Such a relatively coarse sugar provides delayed dissolution, and a dense particle structure, and thus may be appropriate in certain formulations or applications.

The gel-forming agent preferably comprises cornstarch (regular and/or waxy), collagen, hydrolyzed collagen, chitosan, or agarose. The absorbent agent may also comprise polylactic acid alone or in combination with other agents. Other absorbent agents may also be used, as are known in the art.

The wound healing powder may be provided in a dissolvable capsule, e.g., a gelatin capsule, which holds at least 0.5 gram of powder, and more preferably 1 gram of powder. Advantageously, the gelatin in the capsule itself serves as part of the gel forming agent, while permitting contact of the powder with the wound.

### DESCRIPTION OF THE INVENTION

### Example 1

A formulation according to the present invention may comprise 25-50% by weight of a finely granulated sugar, e.g., 5×-10×, 3-50% by weight cornstarch, and 5-50% hydrolyzed collagen and/or powdered collagen. Other ingredients, such as calcium carbonate, magnesium carbonate, or other minerals and/or vitamins may be provided. In some cases, a high activity antibiotic may be added, such as neomycin/bacitracin/polymyxin.

A preferred formulation will self-produce a gel matrix covering the open wound, and will progressively absorb a quantity of wound secretions while remaining adherent to the wound as a cake. Inside the cake, the osmotic pressure remains sufficiently high to render the material at least bacteriostatic, while being generally non-toxic and non-irritating to the patient. Thus, the powder will increasingly gel, without external seeping, and permit a gauze or other covering to remain dry. This, in turn, permits the covering to be replaced without disturbing the wound, to permit fragile new skin to grow uninterrupted. The gel matrix itself is biocompatible, and can be absorbed into the forming granuloma without scarring.

The covering, in this case, is optional, and if present can be a simple cotton gauze pad. A non-stick barrier may also be provided under the pad, especially if the pad will be maintained in position through saturation with secretions.

Thus, while a traditional self-adherent style bandage maybe employed, a sufficient amount of dry powder is applied to retain a non-wetted external portion after application. Thus, one reason for a covering is to retain the dry powder in place.

When the dressing is applied without a covering, only the portion which is promptly hydrated will tend to adhere to the wound, limiting the amount of powder that can be applied and maintained. However, as the wound oozes secretions, dry powder may be added to a moist surface. On the other hand, the surface of a thick powder cake can be wet with a mist of water or the like, leading to formation of a shell which encapsulates a dry reservoir.

It is noted that cornstarch tends to be “waxy”, and tends to interact with water droplets or surfaces by spreading a thin film of powder over the surface, preventing further wetting for an extended period. Thus, cornstarch, while increasing flowability of the dry powder, and forming a matrix when wet, tends to produce a barrier to wetting in the bulk powder. Therefore, the preferred embodiment comprises a component which is additional to the sugar, which is readily wettable and/or hygroscopic, and which retains water, such as collagen powder or hydrolyzed collagen. These materials, which may also form gels, tend to increase flexibility and reduce cracking of the cake when subject to strain. Indeed, in some embodiments, a starch component may be dispensed with, and the formulation may comprise a sugar component and, for example, a peptide component or mucopolysaccharide component. Typically, ingredients which are not broken down and absorbed in an active granuloma are to be avoided in the formulation. When administered, solid physical barriers which prevent direct contact between the powder and the wound surface are also generally avoided.

As discussed above, honey may have beneficial wound healing properties. Honey may be formulated as a powder directly, co-drying it with sugar and then powdering, or mixing honey with starch, e.g., cornstarch, and powdering that mixture.

The resulting product is a powder which, when applied to a wound, absorbs wound secretions and acts as an antibacterial, likely due to the action of a high concentration of sugar. The product promotes healing, and aids in tissue regeneration. The power is applied to a wound in sufficient quantities to cake on the surface, forming a self-adherent dressing. The starch component or another component acts as a matrix for tissue regeneration and a gel medium to provide physical support for the dressing.

Wettability is a measure of the ability of a powder to be wetted with water at a given temperature. Wettability depends on the surfaces of the agglomerates or single particles—are they water repellent or will they absorb water too quickly thus forming a film through which the water cannot penetrate. Generally speaking, wetting is a process in which the gaseous phase at the surface of the solid phase is replaced by a liquid phase, all three phases coexisting for some time, so that a certain amount of intermixtures and solutions (mainly of the solid and the liquid phase) is not only possible but usually unavoidable. The factor deciding if there will be any wetting at all is the interfacial tension between the particle surface and the water. Some particles are wetted easily, and this may depend on the presence or absence of exposed hydrophobic domains. Both finely powdered sugar in an amorphous phase and protein absorb water readily. The water repellence of particles may be overcome, and an interfacial tension facilitating the wetting may be achieved by adding a surface active agent to the surface. Phospholipids such as lecithin may be used, since these are non-toxic, and readily available. The lecithin may be derived from soy, corn and/or egg, for example, and may be hydrogenated. The lecithin is present in a small amount, e.g., <0.1%-1.0%.

The use of a sufficient amount of a surfactant increases the speed of powder wetting, and thus the rate of liquid absorption by the powder. It also improves uniformity of wetting.

The product can be provided as a free powder, to be applied directly to a wound, with a spatula or the like, a scoop, or by sprinkling. In the case of anal fissures and proctitis, or the like, the powder may be applied within a capsule. The capsule is, for example, a standard gelatin capsule. As the gelatin dissolves, the powder is made available to cake with the wound secretions.

The starch may be, for example, cornstarch, which contains amylase and amylopectin, or other polysaccharides. In some cases, waxy maize starch (amylopectin) is employed.

As an alternate to a starch, a carbohydrate formed from saccharide derivatives, such as sulfonated, acetylated, aminated, and other known biocompatible derivatives of saccharide monomers which form biocompatible polymers. For example, chitin or chitosan, algin, and/or agarose, may be employed. Gel forming polymers typically interact with a cross linking agent. Suitable cross linking agents include, for example, calcium salts, such as calcium acetate, for example forming calcium alginate with algin. Organic cross linking agents may also be used.

The disaccharide and polysaccharide (and/or polylactose) combination increases the blood flow within the tissues, which is important for tissue regeneration and aids in promoting a functional microvasculature, and ultimately, successful healing. Preferred sugar components are 5×, 6× or 10× powdered (confectioner's) sugar (sucrose).

The powder may be dispensed from a dry powder container having a set of holes or apertures, by, for example, shaking the powder from a plastic bottle onto the wound. A spatula or the like is used to gently work the powder into the wound secretions, to form a cake. The container can be sealed after a portion of the powder is dispensed, so that the container may be used for multiple applications.

Alternately, a sealed bag or pouch may be provided with a tear-off top for single use administration. For example, a sealed bag or pouch may contain 0.5-1 gm. of powder. The bag or pouch may be paper, foil, aluminized plastic film, plastic, or the like. It is preferred that the bag or pouch be water and moisture resistant, to preserve the powder form during humid conditions.

### Example 2

A capsule is provided containing an individual dose of powder, i.e., 0.5-1 gm. The capsule may be a two-part O (0.68 ml), OO (0.95 ml) or OOO (1.37 ml) size gelatin capsule, for example. The powder can be applied to various kinds of wounds, including ulcers, burns, avulsions, lacerations, surgical excisions, pilonodal cysts, and the like.

The powder is applied to wounds as necessary to absorb the secretions, for example at least twice daily. In a number of tests, with healing is visibly promoted within three days, and complete healing is generally observed within 7-10 days. In the case of open wounds, e.g., pilonidal operations, a longer period of treatment may be necessary.

In the case of a rectal suppository, the capsule is inserted in the rectum after bowel movements, and at least twice daily.

### Example 3

A dressing may be placed over the powder, for example a Telfa dressing. Telfa consists of a thin layer of absorbent cotton fibers, enclosed in a sleeve of poly(ethylene terephthalate) that is perforated in a regular pattern and sealed along two edges. The plastic film is present to prevent the dressing adhering to the surface of the wound, and is perforated to allow the passage of exudate from the wound into the body of the pad.

The powder may be provided, for example, within a self-dissolving gelatin or agarose pouch, adhered to the dressing, with or without the Telfa film, beneath the dressing, which can be cotton gauze, non-woven fibers, or other medically acceptable material. That is, the powder is captured within a dissolving film, which, when moistened by the wound secretions, wets the powder, causing the osmotic component to dissolve, and the gel forming component (which may indeed include the dissolving film material) to gel.

According to the present invention, since the starch absorbs the secretions, the body of the pad serves only a secondary function, and the combined dressing may be used on wounds which produce secretions which would normally be contraindicated for Telfa® alone.

### Example 4

A spray or jet of powder suspended in a volatile carrier liquid may be employed. In general, the carrier is non-aqueous (though need not be completely anhydrous), non-stinging, non-toxic liquid that rapidly vaporizes leaving the powder adhered to the wound secretions.

The liquid may be self-pressurizing, pump-spray, or compressed by another gas such as carbon dioxide, nitrous oxide, or nitrogen.

It is desirable that the active powder ingredients be suspended in a volatile liquid carrier so that this suspension may be sprayed onto the wound. This technique is desirable for two reasons. First, the presence of the volatile liquid provides a surface tension that adheres the powder in a dense film to the wound, and second, the presence of the volatile liquid controls the undesirable tendency of the active powder to form a cloud of dust. The quantity of the carrier should be sufficient to accomplish its objectives but not so great as to drip or run from the site of application. The ratio of carrier to powder solids may vary from about 10 to 1 to 1:0.5 by weight.

Preferably, the suspended powders are very fine, in order to avoid clogging the nozzle. Thus, 10× or 12× sugar, and fine cornstarch powder, are preferred. A preferred propellant is isobutane/propane. A preferred volatile liquid is n-pentane.

Many organic substances or mixtures thereof, may serve as the volatile liquid.

The primary considerations in the selection of such a liquid are the relative toxicity of the vapor and the irritancy of the liquid to the user's skin or wound. Other important considerations in selecting a volatile liquid are the flammability of such liquid and the exact degree of volatility. While obviously, the technology is operative even if a highly flammable substance is used as the volatile liquid, it is preferable either to avoid the use of highly flammable liquids, or to use them in conjunction with non-flammable liquids Likewise, ozone depleting potential and global warming potential are likewise considerations.

Liquids with boiling points ranging from about −30° to about 82° C. may be used as the volatile liquid in conjunction with the chitin powder. Compounds with suitable properties include esters such as ethyl acetate and methyl acetate; ketones such as methyl ethyl ketone and acetone; hydrocarbons, such as the straight chained alkanes, butanes, pentane and hexane; cyclic hydrocarbons such as cyclopropane, cyclobutane, cyclopentane, and cyclohexane; branched chain hydrocarbons such as 2,2-dimethyl propane, the methyl pentanes, the dimethyl pentanes and the dimethyl butanes; alcohols, such as ethyl alcohol and iso-propyl alcohol, ethers, such as dimethyl ether, diethyl ether, diisopropyl ether, the methyl ethers of ethyl, isopropyl, propyl, n-butyl, t-butyl and isobutyl alcohols, and the ethyl ethers of n-propyl, isopropyl, isobutyl, t-butyl and 2-butyl alcohols; and non-toxic refrigerant gasses which are provided in such manner as to avoid surface freezing or frostbite. With respect to ethanol, due to distribution issues, preferred forms are denatured, e.g., SD Alcohol 3-A, SD Alcohol 30, SD Alcohol 39-B, SD Alcohol 39-C, SD Alcohol 40-B, or SD Alcohol 40-C, denatured with t-Butyl Alcohol, Denatonium Benzoate, Quassin, Brucine and Brucine Sulfate. Diethyl Phthalate, or Methyl Alcohol.

As noted above, the volatile liquid may consist of either a single pure material or a combination of materials. It is preferred that liquids at either extreme of the boiling point range not be used above, but rather as part of a mixture of substances acting as a carrier. As noted above, in selecting a carrier substance, consideration should be given to the fire hazard which the substance might present. Thus, the ethers and hydrocarbons are rather hazardous materials, while the alkyl fluorides present no fire hazard. It is preferred that the volatile liquid contain a non-flammable fluorocarbon or mixture thereof if the highly flammable hydrocarbons or ethers are used.

The powder may be applied either from aerosol cans, or from a hand operated spray device. If the dry powder is sprayed from an aerosol can, a propellant substance must be used. The choice of propellant material is not critical and any nontoxic substance which develops the requisite pressure may be used to perform the essentially mechanical function of driving the volatile liquid and the powder out of the aerosol container. All propellants which are commonly used in aerosol cans are, of course, suitable for use in this application. Low boiling substances suitable for use as the volatile liquid may also serve as a propellant.

See, U.S. Pat. No. 4,035,267, expressly incorporated herein by reference.

When applied as a volatile liquid, it may be advantageous for one or more components of the composition to be partially or completely soluble in the carrier, resulting in an emulsion. As the volatile liquid dries, this will form a film, which entraps the particulate (insoluble) phase, which will be distributed evenly due to the aerosol action. The film will be generally hygroscopic, and absorb the wound secretions. An antibiotic may also be provided in the suspension or solution. If the wound does not have sufficient secretions, the skin may be wetted with a water film before spraying. A biocompatible polymer may be provided in the suspension (or solution) which assist in the formation of the hygroscopic swelling film. For example, the film-forming agent may be one or more polymers selected from the group consisting of film forming plastic material selected from the group consisting of N-butyl methacrylate, isobutyl methacrylate, 2-ethoxyethyl methacrylate, methyl methacrylate, 2-ethoxyethyl methacrylate/methyl methacrylate (90/10 copolymer), N-butyl methacrylate/isobutyl methacrylate (50/50 copolymer), nitrocellulose, or N-butyl methacrylate/methyl methacrylate (80/20 copolymer). See, U.S. Pat. No. 3,932,602, U.S. Pat. No. 3,928,556, and U.S. 2007/0048355, expressly incorporated herein by reference.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A method for treating wounds, comprising:
providing a dry powder mixture, which when partially hydrated, forms a gel having an osmotic pressure sufficient to achieve and maintain antiseptic conditions in hydrated portions of the gel, the gelled portion comprising a biocompatible polymer, the dry powder being readily wettable by wound secretions; and
applying the dry powder to a wound having wound secretions in sufficient excess to produce a self-adherent gel cake having an exposed dry powder surface.

2. The method according to claim 1, wherein the dry powder further comprises polylactic acid.

3. The method according to claim 1, wherein the dry powder comprises finely powdered sucrose.

4. The method according to claim 1, wherein the dry powder comprises a powdered form of honey.

5. The method according to claim 1, wherein the gel comprises calcium alginate.

6. The method according to claim 1, wherein the dry powder comprises collagen.

7. The method according to claim 1, wherein the dry powder comprises hydrolyzed collagen.

8. The method according to claim 1, wherein the dry powder comprises gelatin.

9. The method according to claim 1, wherein the dry powder comprises agarose.

10. The method according to claim 1, wherein the dry powder comprises chitosan.

11. The method according to claim 1, wherein the dry powder comprises cornstarch.

12. The method according to claim 1, wherein the dry powder comprises a surfactant.

13. The method according to claim 1, wherein the dry powder comprises lecithin.

14. The method according to claim 1, wherein the dry powder comprises cornstarch, hydrolyzed collagen, and sucrose.

15. The method according to claim 13, wherein the dry powder further comprises lecithin.

16. The method according to claim 1, wherein the dry powder comprises about 15-50% by weight sucrose, 5-50% by weight cornstarch, 5-50% by weight collagen, and less than 1% by weight lecithin.

17. The method according to claim 1, wherein the dry powder comprises an antibiotic.

18. A dry pharmaceutically acceptable powder adapted for application to wounds, comprising:
a powdered biocompatible polymer gel-forming agent;
a non-irritating, non-ionic osmotic agent; and
a surfactant,
wherein the dry powder when partially hydrated forms a gel having an osmotic pressure sufficient to achieve and maintain antiseptic conditions in hydrated portions of the gel, the dry powder being readily wettable by wound secretions.

19. The powder according to claim 18, further comprising a cross linking agent adapted to cross link the biocompatible polymer gel-forming agent to form a gel when hydrated.

20. The powder according to claim 18, wherein the biocompatible gel-forming agent comprises agarose and cornstarch, the cross linking agent comprises calcium ions, the non-irritating, non-ionic osmotic agent comprises finely powdered sucrose, and the surfactant comprises lecithin.

21. A method for healing a wound on skin or mucous membranes, comprising:
providing a pharmaceutically acceptable dry powder comprising:
(a) a sugar selected from the group consisting of one or more pharmaceutically acceptable monosaccharides and disaccharides, in an amount sufficient to exert an antiseptic effect;
(b) a gel-forming composition which forms a biocompatible polymer gel matrix with wound secretions; and
(c) a surfactant,
administering the pharmaceutically acceptable dry powder to a wound on skin or mucous membranes having wound secretions associated therewith, in sufficient quantity to form a self-adherent gel on the wound with a dry powder surface, having an osmotic tension sufficient to achieve an antiseptic effect.

22. The method according to claim 21, wherein the sugar comprises at least 20% by weight of the free dry powder.

23. The method according to claim 21, wherein the sugar comprises honey.

24. The method according to claim 21, wherein the biocompatible polymer gel matrix formed with wound secretions is permeable to permit atmospheric oxygen to reach the wound.
